(19) United States
(12) Patent Application Publication
(10) Pub. No.: US 2014/0121421 A1
(43) Pub. Date: May 1, 2014

(54) Hydrogenation Reaction Conditions To Produce Ethanol and Reduce Ethyl Acetate Formation

(71) Applicant: CELANESE INTERNATIONAL CORPORATION, Irving, TX (US)

(72) Inventors: Radmila Wollrab, Pasadena, TX (US); Victor J. Johnston, Houston, TX (US); Heiko Weiner, Pasadena, TX (US)

(73) Assignee: CELANESE INTERNATIONAL CORPORATION, Irving, TX (US)

(21) Appl. No.: 13/661,543

(22) Filed: Oct. 26, 2012

(51) Int. Cl. C07C 29/149

(52) U.S. Cl. CPC C07C 29/149
USPC 568/885

(57) ABSTRACT

The present invention is directed to processes for selective formation of ethanol from acetic acid by hydrogenating acetic acid in the presence of a hydrogenation catalyst, wherein the temperature and liquid hourly space velocity (LHSV) are controlled to maximize acetic acid conversion and to minimize selectivity to ethyl acetate. The hydrogenation catalyst comprises a metal selected from the group consisting of platinum, palladium, gold, iridium, osmium, and rhodium on a support modified by at least one support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof.

### FIELD OF THE INVENTION

The present invention relates to processes for producing ethanol from a feed stream comprising a carboxylic acid in the presence of hydrogenation catalysts. In particular, the present invention relates to a process for producing ethanol from a feed stream with a temperature of greater than 225° C. and a liquid hourly space velocity of at least 0.3 hr⁻¹.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulosic materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulosic material, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The reduction of various carboxylic acids over metal oxides has been proposed by EP0175558 and U.S. Pat. No. 4,398,039. A summary some of the developmental efforts for hydrogenation catalysts for conversion of various carboxylic acids is provided in Yokoyama, et al., “Carboxylic acids and derivatives” in: Fine Chemicals Through Heterogeneous Catalysis, 2001, 370-379.

U.S. Pat. No. 6,495,730 describes a process for hydrogenating carboxylic acid using a catalyst comprising activated carbon to support active metal species comprising ruthenium and tin. U.S. Pat. No. 6,204,417 describes another process for preparing aliphatic alcohols by hydrogenating aliphatic carboxylic acids or anhydrides or esters thereof or lactones in the presence of a catalyst comprising Pt and Re. U.S. Pat. No. 5,149,680 describes a process for the catalytic hydrogenation of carboxylic acids and their anhydrides to alcohols and/or esters in the presence of a catalyst containing a Group VIII metal, such as palladium, a metal capable of alloying with the Group VIII metal, and at least one of the metals rhenium, tungsten or molybdenum. U.S. Pat. No. 4,777,303 describes a process for the productions of alcohols by the hydrogenation of carboxylic acids in the presence of a catalyst that comprises a first component which is either molybdenum or tungsten and a second component which is a noble metal of Group VIII on a high surface area graphitized carbon. U.S. Pat. No. 4,804,791 describes another process for the production of alcohols by the hydrogenation of carboxylic acids in the presence of a catalyst comprising a noble metal of Group VIII and rhenium. U.S. Pat. No. 4,517,391 describes preparing ethanol by hydrogenating acetic acid under superatmospheric pressure and at elevated temperatures by a process wherein a predominantly cobalt-containing catalyst.

Existing processes suffer from a variety of issues impeding commercial viability including: (i) catalysts without requisite selectivity to ethanol; (ii) catalysts which are possibly prohibitively expensive and/or nonselective for the formation of ethanol and that produce undesirable by-products; (iii) required operating temperatures and pressures which are excessive; and/or (iv) insufficient catalyst life.

### SUMMARY OF THE INVENTION

The present invention relates to processes for hydrogenating acetic acid to make ethanol at high selectivities. In a first embodiment, the invention is directed to a process for producing ethanol, comprising contacting acetic acid and hydrogen in a reactor at a temperature of greater than 225° C. in the presence of a catalyst, under conditions effective to form ethanol, wherein the liquid hourly space velocity (LHSV) of the acetic acid fed to the reactor is from 0.3 hr⁻¹ to 1.6 hr⁻¹, wherein the catalyst comprises at least one metal selected from the group consisting of platinum, palladium, gold, iridium, osmium, and rhodium, on a support modified by a support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group 111B metal oxides, (viii) Group IIIB metal metasilicates, and mixtures thereof. Acetic acid selectivity to ethanol may be greater than 60%. The catalyst may further comprise at least one active metal selected from the group consisting of copper, iron, cobalt, vanadium, nickel, titanium, zinc, chromium, tin, lanthanum, cerium and manganese. In one embodiment the catalyst does not contain rhenium, ruthenium, molybdenum, and/or tungsten. In some embodiments, the catalyst comprises platinum and tin. The support modifier may be selected from the group consisting of oxides and metasilicates of sodium, potassium, magnesium, calcium, scandium, yttrium, and zinc. The support material may be selected from the group consisting of silica, alumina, titania, silica/alumina, pyrogenic silica, high purity silica, zirconia, carbon, zeolites and mixtures thereof. The acetic acid may be derived from a carbonaceous material selected from the group consisting of oil, coal, natural gas and biomass. In some embodiments, the temperature is from 250° C. to 300° C., the LHSV of the acetic acid fed to the reactor is at least 0.6 hr⁻¹, e.g., from 0.6 to 1.6 hr⁻¹. In some embodiments, the temperature is at least 250° C., the LHSV of the acetic acid fed to the reactor is at least 0.5 hr⁻¹, e.g., from 0.5 to 1.6 hr⁻¹, and conversion of acetic acid is at least 15%. In another embodiment, the temperature is at least 275° C., the LHSV of the acetic acid fed to the reactor is at least 0.6 hr⁻¹, e.g., from 0.6 to 1.6 hr⁻¹, and conversion of acetic acid is at least 20%. In yet another embodiment, the temperature is at least 300° C., the LHSV of the acetic acid fed to the reactor is at least 1.2 hr⁻¹, e.g., from 1.2 to 1.6 hr⁻¹, and conversion of acetic acid is at least 30%.

In a second embodiment, the invention is directed to a process for producing ethanol, comprising contacting acetic acid and hydrogen in a reactor at a temperature from 225° C. to 300° C. in the presence of a catalyst, under conditions effective to form ethanol wherein the liquid hourly space velocity of the acetic acid fed to the reactor is at least 0.3 to 1.6 hr⁻¹, wherein the catalyst comprises a first metal selected from the group consisting of platinum, palladium, gold, iridium, osmium, and rhodium and a second metal selected from the group consisting of copper, iron, cobalt, vanadium, nickel, titanium, zinc, chromium, tin, lanthanum, cerium and manganese on a support. The support may comprises a support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates. The support modifier may be selected from the group consisting of oxides and metasilicates of sodium, potassium, magnesium, calcium, scandium, yttrium, and zinc. The support material may be selected from the group consisting of silica, alumina, titania, silica/alumina, pyrogenic silica, high purity silica, zirconia, carbon, zeolites and mixtures thereof. The acetic acid may be derived from a carbonaceous material selected from the group consisting of oil, coal, natural gas and biomass. In some embodiments, the temperature is from 250° C. to 300° C., the LHSV of the acetic acid fed to the reactor is at least 0.6 hr⁻¹, e.g., from 0.6 to 1.6 hr⁻¹. In some embodiments, the temperature is at least 250° C., the LHSV of the acetic acid fed to the reactor is at least 0.5 hr⁻¹, e.g., from 0.5 to 1.6 hr⁻¹, and conversion of acetic acid is at least 15%. In another embodiment, the temperature is at least 275° C., the LHSV of the acetic acid fed to the reactor is at least 0.6 hr⁻¹, e.g., from 0.6 to 1.6 hr⁻¹, and conversion of acetic acid is at least 20%. In yet another embodiment, the temperature is at least 300° C., the LHSV of the acetic acid fed to the reactor is at least 1.2 hr⁻¹, e.g., from 1.2 to 1.6 hr⁻¹, and conversion of acetic acid is at least 30%.

### BRIEF DESCRIPTION OF DRAWINGS

The invention is described in detail below with reference to the appended non-limiting figures, in which:

FIG. 1 shows acetic acid conversion for one embodiment of the present invention.

FIG. 2 shows ethyl acetate selectivity for one embodiment of the present invention.

### DETAILED DESCRIPTION OF THE INVENTION

The present invention relates to processes for recovering ethanol produced by hydrogenating a feed stream comprising a carboxylic acid, e.g., acetic acid in the presence of a catalyst. The catalyst comprises at least one metal selected from the group consisting of platinum, palladium, gold, iridium, osmium, and rhodium, on a support modified by a support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In one embodiment, the catalyst comprises platinum and tin on a silicaceous support modified by calcium metasilicate.

The hydrogenation reaction produces a crude ethanol stream that comprises ethanol, water, ethyl acetate, acetaldehyde, and other impurities. The processes of the present invention surprisingly are able to minimize selectivity to ethyl acetate and maximize selectivity to ethanol by controlling the reaction temperature and/or the liquid hourly space velocity (LHSV) of the feed stream. Preferably, the selectivity to ethyl acetate is less than the selectivity to ethanol. Reducing the ethyl acetate formation advantageously increases the production of ethanol, and decreases the energy and capital required to purify the crude ethanol stream. In addition, these controls on the hydrogenation process allow operators to manage production of different products by adjusting the temperature and LHSV of the feed stream.

Selectivity is expressed as a mole percent based on converted acetic acid. For purposes of the present invention, the term “conversion” refers to the amount of acetic acid that is converted to a compound other than acetic acid. The acetic acid conversion may be at least 10%, e.g., at least 15%, at least 20%, or at least 30%. It should be understood that each compound converted from acetic acid has an independent selectivity and that selectivity is independent of conversion. For example, if 60 mole % of the converted acetic acid is converted to ethanol, we refer to the ethanol selectivity as 60%. Preferably, the selectivity to ethanol is at least 60%, e.g., at least 70%, or at least 80%, at least 85% or at least 88%. In general, the selectivity of acetic acid to ethyl acetate may be less than 25%, e.g., less than 20%, less than 18%, or less than 15%. The reaction temperature may be from 225° C. to 300° C., e.g., from 250° C. to 300° C. The ethyl acetate selectivity may be reduced by decreasing the temperature of the reaction by at least 5° C., e.g., at least 15° C., or least 25° C. Preferably, the reaction temperature is not reduced below 225° C. At these reaction temperatures, the ethyl acetate selectivity may also be reduced by controlling the LHSV of the feed stream to the reactor within a range from 0.3 hr⁻¹ to 1.6 hr⁻¹, e.g., 0.5 hr⁻¹ to 1.6 hr⁻¹, 1 hr⁻¹ to 1.6 hr⁻¹, or 1 hr⁻¹ to 1.3 hr¹. Above 1.6 hr¹ the acetic acid conversion tends to drop below 10% and below 0.3 hr¹ the ethyl acetate selectivity tends to be above 20%. Operating above 1 hr¹ may provide a greater decrease in ethyl acetate selectivity with slightly lower acetic acid conversions. The lower acetic acid conversions may be tolerated using a larger acetic acid recycle and the overall process benefits by reducing the amount of ethyl acetate that needs to be purged from the system. Thus, to achieve suitable acetic acid conversion and ethyl acetate selectivity the temperature of the reactor and/or LHSV is controlled. In one embodiment of the present invention, the ethyl acetate selectivity may be reduced by decreasing the temperature of the reaction and increasing the LHSV.

The LHSV may be controlled for various reactor temperatures to achieve reductions in ethyl acetate selectivity, while maintaining acetic acid conversions of greater than 10%. In one embodiment, when the reactor temperature is at least 225° C. the LHSV may from 0.3 to 0.6 hr⁻¹. At these lower temperatures, the acetic acid conversion is less than 10% above 0.6 hr⁻¹.

In another embodiment, the reactor temperature is at least 250° C. and the LHSV of the feed stream is from 0.5 hr⁻¹ to 1.6 hr⁻¹, e.g., from 1 hr⁻¹ to 1.6 hr⁻¹ or from 1.2 hr⁻¹ to 1.6 hr⁻¹. In yet another embodiment, the temperature in the reactor is at least 275° C. and the LHSV of the feed stream is 0.6 hr⁻¹ to 1.6 hr⁻¹, e.g., from 0.8 hr⁻¹ to 1.6 hr⁻¹ or from 1 hr⁻¹ to 1.6 hr⁻¹. In still other embodiments, the temperature in the reactor is at least 300° C. and the LHSV of the feed stream is from 1.2 hr⁻¹ to 1.6 hr⁻¹, e.g., from 1.3 hr⁻¹ to 1.6 hr⁻¹ or from 1.4 hr⁻¹ to 1.6 hr⁻¹. At temperatures higher than 300° C. the ethyl acetate selectivity may be greater than 20%. Higher temperatures may provide higher conversions of acetic acid. For example, above 250° C. the acetic acid conversion may be greater than 15%, above 275° C. the acetic acid conversion may be greater than 20%, and above 300° C. the acetic acid conversion may be greater than 30%.

### Acetic Acid Hydrogenation

In one embodiment there is a process for producing ethanol by hydrogenating feedstock comprising compounds selected from the group consisting of acetic acid, ethyl acetate and mixtures thereof in the presence of the catalyst. One particular preferred reaction is to make ethanol from acetic acid. The hydrogenation reaction may be represented as follows:

HOAc+2H₂→EtOH+H₂O

In some embodiments, the catalyst may be characterized as a bifunctional catalyst in that it effectively catalyzes the hydrogenation of acetic acid to ethanol as well as the conversion of ethyl acetate to one or more products, preferably ethanol.

The raw materials, acetic acid and hydrogen, fed to the reactor used in connection with the process of this invention may be derived from any suitable source including natural gas, petroleum, coal, biomass, and so forth. As examples, acetic acid may be produced via methanol carbonylation, acetaldehyde oxidation, ethane oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from other carbon sources have drawn increasing interest. In particular, when petroleum is relatively expensive, it may become advantageous to produce acetic acid from synthesis gas (“syngas”) that is derived from other available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol stream may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. Black liquor, which is an aqueous solution of lignin residues, hemicellulose, and inorganic chemicals, may also be used as a biomass source. Biomass-derived syngas has a detectable ¹⁴C isotope content as compared to fossil fuels such as coal or natural gas.

In another embodiment, the acetic acid used in the hydrogenation step may be formed from the fermentation of biomass. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference.

The acetic acid fed to the hydrogenation reactor may also comprise acetic anhydride, acetaldehyde, ethyl acetate, propionic acid, water, and mixtures thereof.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

The reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles. In some embodiments, multiple catalyst beds are employed in the same reactor or in different reactors, e.g., in series. For example, in one embodiment, a first catalyst functions in a first catalyst stage as a catalyst for the hydrogenation of a carboxylic acid, e.g., acetic acid, to its corresponding alcohol, e.g., ethanol, and a second bifunctional catalyst is employed in the second stage for converting unreacted acetic acid to ethanol as well as converting byproduct ester, e.g., ethyl acetate, to additional products, preferably to ethanol. The catalysts of the invention may be employed in either or both the first and/or second stages of such reaction systems.

The hydrogenation in the reactor may be carried out in either the liquid phase or vapor phase. Preferably, the reaction is carried out in the vapor phase under the following conditions. The reaction temperature may range from 225° C. to 300° C., e.g., from 250° C. to 300° C., from 250° C. to 275° C. The pressure may range from 10 kPa to 3000 kPa, e.g., from 50 kPa to 2300 kPa, or from 100 kPa to 2000 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

In one embodiment, the hydrogenation process may also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

In various embodiments of the present invention, the crude ethanol product produced by the reactor, before any subsequent processing, such as purification and separation, will typically comprise unreacted acetic acid, ethanol and water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 15 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.1 wt. % to 15 wt. %, from 0.1 wt. % to 10 wt. % or from 0.1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

An ethanol product may be recovered from the crude ethanol product produced by the reactor using the catalyst of the present invention may be recovered using several different techniques.

The ethanol product may be an industrial grade ethanol comprising from 75 to 96 wt. % ethanol, e.g., from 80 to 96 wt. % or from 85 to 96 wt. % ethanol, based on the total weight of the ethanol product. The industrial grade ethanol may have a water concentration of less than 12 wt. % water, e.g., less than 8 wt. % or less than 3 wt. %. In some embodiments, when further water separation is used, the ethanol product preferably contains ethanol in an amount that is greater than 96 wt. %, e.g., greater than 98 wt. % or greater than 99.5 wt. %. The ethanol product having further water separation preferably comprises less than 3 wt. % water, e.g., less than 2 wt. % or less than 0.5 wt. %.

The finished ethanol composition produced by the embodiments of the present invention may be used in a variety of applications including fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In the production of ethyl acetate, the finished ethanol composition may be esterified with acetic acid. In another application, the finished ethanol composition may be dehydrated to produce ethylene. Any known dehydration catalyst, such as zeolite catalysts or phosphotungstic acid catalysts, can be employed to dehydrate ethanol, as described in U.S. Pub. Nos. 2010/0030002 and 2010/0030001 and WO2010146332, the entire contents and disclosures of which are hereby incorporated by reference.

### Catalyst Composition

### Active Metals

One or more active metals may also be impregnated on the support. In one embodiment, the one or more active metals are selected from the group consisting of copper, iron, cobalt, nickel, rhodium, platinum, palladium, osmium, iridium, titanium, zinc, chromium, tin, lanthanum, cerium, manganese, and gold. The total weight of all the active metals present in the catalyst preferably is from 0.1 to 25 wt. %, e.g., from 0.1 to 15 wt. %, or from 0.1 wt. % to 10 wt. %. In one embodiment, the catalyst does not comprise ruthenium, rhenium, molybdenum, tungsten, or mixtures thereof. For purposes of the present specification, unless otherwise indicated, weight percent is based on the total weight the catalyst including metal and support.

In some embodiments, the catalyst contains at least two active metals. A first active metal may be selected from the group consisting of platinum, palladium, gold, iridium, osmium, and rhodium. When the first metal comprises platinum, palladium, gold, iridium, osmium, and rhodium, it is preferred that the catalyst comprises such metals in an amount less than 5 wt. %, e.g., less than 3 wt. % or less than 1 wt. %, due to their precious nature. A second active metal, which is different than the first metal, is selected from the group consisting of copper, iron, cobalt, vanadium, nickel, titanium, zinc, chromium, tin, lanthanum, cerium and manganese. Additional active metals may also be used in some embodiments.

Preferred bimetallic combinations for some exemplary catalyst compositions include platinum/tin, cobalt/palladium, cobalt/platinum, cobalt/chromium, cobalt/tin, silver/palladium, copper/palladium, copper/zinc, nickel/palladium, and gold/palladium. Additional metal combinations may include platinum/tin/palladium, platinum/tin/cobalt, platinum/tin/copper, platinum/tin/chromium, platinum/tin/zinc, and platinum/tin/nickel.

When the catalyst comprises two or more active metals, e.g., a first active metal and a second active metal, the first active metal may be present in the catalyst in an amount from 0.1 to 10 wt. %, e.g. from 0.1 to 5 wt. %, or from 0.1 to 3 wt. %. The second active metal may be present in an amount from 0.1 to 20 wt. %, e.g., from 0.1 to 10 wt. %, or from 0.1 to 5 wt. %. For catalysts comprising two or more active metals, the metals may be alloyed with one another or may comprise a non-alloyed metal solution or mixture.

The preferred metal ratios may vary somewhat depending on the active metals used in the catalyst. In some embodiments, the mole ratio of the first active metal to the second active metal preferably is from 10:1 to 1:10, e.g., from 4:1 to 1:4, from 2:1 to 1:2, from 1.5:1 to 1:1.5 or from 1.1:1 to 1:1.1.

### Support Materials

The catalysts of the present invention may be on any suitable support material. In one embodiment, the support material may be an inorganic oxide. In one embodiment, the support material may be selected from the group consisting of silica, alumina, titania, silica/alumina, calcium metasilicate, pyrogenic silica, high purity silica, zirconia, carbon, zeolites and mixtures thereof. Preferably, the support material comprises silica. In preferred embodiments, the support material is present in an amount from 25 wt. % to 99 wt. %, e.g., from 30 wt. % to 98 wt. % or from 35 wt. % to 95 wt. %.

The surface area of silicaceous support material, e.g., silica, preferably is at least about 50 m²/g, e.g., at least about 100 m²/g, at least about 150 m²/g, at least about 200 m²/g or most preferably at least about 250 m²/g. In terms of ranges, the silicaceous support material preferably has a surface area of from 50 to 600 m²/g, e.g., from 100 to 500 m²/g or from 100 to 300 m²/g. High surface area silica, as used throughout the application, refers to silica having a surface area of at least about 250 m²/g. For purposes of the present specification, surface area refers to BET nitrogen surface area, meaning the surface area as determined by ASTM D6556-04, the entirety of which is incorporated herein by reference.

The silicaceous support material also preferably has an average pore diameter of from 5 to 100 nm, e.g., from 5 to 30 nm, from 5 to 25 nm or from about 5 to 10 nm, as determined by mercury intrusion porosimetry, and an average pore volume of from 0.5 to 2.0 cm³/g, e.g., from 0.7 to 1.5 cm³/g or from about 0.8 to 1.3 cm³/g, as determined by mercury intrusion porosimetry.

The morphology of the support material, and hence of the resulting catalyst composition, may vary widely. In some exemplary embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the silicaceous support material has a morphology that allows for a packing density of from 0.1 to 1.0 g/cm³, e.g., from 0.2 to 0.9 g/cm³ or from 0.5 to 0.8 g/cm³. In terms of size, the silica support material preferably has an average particle size, e.g., meaning the diameter for spherical particles or equivalent spherical diameter for non-spherical particles, of from 0.01 to 1.0 cm, e.g., from 0.1 to 0.5 cm or from 0.2 to 0.4 cm. Since the one or more active metal(s) that are disposed on or within the support are generally very small in size, those active metals should not substantially impact the size of the overall catalyst particles. Thus, the above particle sizes generally apply to both the size of the support as well as to the final catalyst particles.

### Other Support Modifiers

The support material may also comprise at support modifier. A support modifier may adjust the acidity of the support material. In one embodiment, the total weight of the support modifiers are present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst.

Support modifiers may adjust the acidity of the support. For example, the acid sites, e.g. Brønsted acid sites, on the support material may be adjusted by the support modifier to favor selectivity to ethanol during the hydrogenation of acetic acid. The acidity of the support material may be adjusted by reducing the number or reducing the availability of Brønsted acid sites on the support material. The support material may also be adjusted by having the support modifier change the pKa of the support material. Unless the context indicates otherwise, the acidity of a surface or the number of acid sites thereupon may be determined by the technique described in F. Delannay, Ed., “Characterization of Heterogeneous Catalysts”; Chapter III: Measurement of Acidity of Surfaces, p. 370-404; Marcel Dekker, Inc., N.Y. 1984, the entirety of which is incorporated herein by reference. In particular, the use of modified supports that adjusts the acidity of the support to make the support less acidic or more basic favors formation of ethanol over other hydrogenation products.

The support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

In one preferred embodiment, the catalyst comprises from 0.25 to 1.25 wt. % platinum and 0.25 to 3 wt. % tin. These preferred active metals are on a silica support. Preferably, the silica support also comprises a support modifier such as CaSiO₃.

One or more support modifiers, if desired, may also be added to the support by mixing or through impregnation. Powdered materials of the modified supports or a precursor thereto may pelletized, crushed and sieved and added to the support. The use of a solvent, such as water, glacial acetic acid, a strong acid such as hydrochloric acid, nitric acid, or sulfuric acid, or an organic solvent, may be preferred. The resulting mixture may be stirred and added to additional support material using, for example, incipient wetness techniques in which the tin is added to a support material having the same pore volume as the volume of the solution. Capillary action then draws the tin into the pores in the support material. The support containing tin can then be formed by drying to drive off water and any volatile components within the support solution and depositing the tin on the support material. Drying may occur, for example, at a temperature of from 50° C. to 300° C., e.g., from 100° C. to 200° C. or about 120° C., optionally for a period of from 1 to 24 hours, e.g., from 3 to 15 hours or from 6 to 12 hours.

Once formed, the modified supports may be shaped into particles having the desired size distribution, e.g., to form particles having an average particle size in the range of from 0.2 to 0.4 cm. The supports may be extruded, pelletized, tabletized, pressed, crushed or sieved to the desired size distribution. Any of the known methods to shape the support materials into desired size distribution can be employed.

In a preferred method of preparing the catalyst, the active metals are impregnated onto the support. A precursor of the first active metal (first metal precursor) preferably is used in the metal impregnation step, such as a water soluble compound or water dispersible compound/complex that includes the first metal of interest. Depending on the metal precursor employed, the use of a solvent, such as water, glacial acetic acid or an organic solvent, may be preferred. The second active metal precursor also preferably is impregnated into the support from a second metal precursor. If desired, a third metal or third metal precursor may also be impregnated into the support.

Impregnation occurs by adding, optionally drop wise, either or both the first metal precursor and/or the second metal precursor and/or additional metal precursors, preferably in suspension or solution, to the dry support. The resulting mixture may then be heated, e.g., optionally under vacuum, in order to remove the solvent. Additional drying and calcining may then be performed, optionally with ramped heating to form the final catalyst composition. Upon heating and/or the application of vacuum, the metal(s) of the metal precursor(s) preferably decompose into their elemental (or oxide) form. In some cases, the completion of removal of the liquid carrier, e.g., water, may not take place until the catalyst is placed into use and calcined, e.g., subjected to the high temperatures encountered during operation. During the calcination step, or at least during the initial phase of use of the catalyst, such compounds are converted into a catalytically active form of the metal or a catalytically active oxide thereof.

Impregnation of the first and second metals (and optional additional metals) into the support may occur simultaneously (co-impregnation) or sequentially. In simultaneous impregnation, the first and second metal precursors (and optionally additional metal precursors) are mixed together and added to the support together, followed by drying and calcination to form the final catalyst composition. With simultaneous impregnation, it may be desired to employ a dispersion agent, surfactant, or solubilizing agent, e.g., ammonium oxalate, to facilitate the dispersing or solubilizing of the first and second metal precursors in the event the two precursors are incompatible with the desired solvent, e.g., water.

In sequential impregnation, the first metal precursor is first added to the support followed by drying and calcining, and the resulting material is then impregnated with the second metal precursor followed by an additional drying and calcining step to form the final catalyst composition. Additional metal precursors (e.g., a third metal precursor) may be added either with the first and/or second metal precursor or a separate third impregnation step, followed by drying and calcination. Of course, combinations of sequential and simultaneous impregnation may be employed if desired.

Suitable metal precursors include, for example, metal halides, amine solubilized metal hydroxides, metal nitrates or metal oxalates. For example, suitable compounds for platinum precursors and palladium precursors include chloroplatinic acid, ammonium chloroplatinate, amine solubilized platinum hydroxide, platinum nitrate, platinum tetra ammonium nitrate, platinum chloride, platinum oxalate, palladium nitrate, palladium tetra ammonium nitrate, palladium chloride, palladium oxalate, sodium palladium chloride, and sodium platinum chloride. Generally, both from the point of view of economics and environmental aspects, aqueous solutions of soluble compounds of platinum are preferred. A particularly preferred precursor to platinum is platinum ammonium nitrate, Pt(NH₃)₄(NO₄)₂. Calcining of the solution with the support and active metal may occur, for example, at a temperature of from 250° C. to 800° C., e.g., from 300 to 700° C. or about 500° C., optionally for a period of from 1 to 12 hours, e.g., from 2 to 10 hours, from 4 to 8 hours or about 6 hours.

In one aspect, the “promoter” metal or metal precursor is first added to the support, followed by the “main” or “primary” metal or metal precursor. Of course the reverse order of addition is also possible. Exemplary precursors for promoter metals include metal halides, amine solubilized metal hydroxides, metal nitrates or metal oxalates. As indicated above, in the sequential embodiment, each impregnation step preferably is followed by drying and calcination. In the case of promoted bimetallic catalysts as described above, a sequential impregnation may be used, starting with the addition of the promoter metal followed by a second impregnation step involving co-impregnation of the two principal metals, e.g., Pt and Sn.

As an example, PtSn/CaSiO₃ on SiO₂ may be prepared by a first impregnation of CaSiO₃ onto the SiO₂, followed by the co-impregnation with Pt(NH₃)₄(NO₄)₂ and Sn(AcO)₂. Again, each impregnation step may be followed by drying and calcination steps. In most cases, the impregnation may be carried out using metal nitrate solutions. However, various other soluble salts, which upon calcination release metal ions, can also be used. Examples of other suitable metal salts for impregnation include, metal acids, such as perrhenic acid solution, metal oxalates, and the like. In those cases where substantially pure ethanol is to be produced, it is generally preferable to use nitrogenous amine and/or nitrate based precursors.

The invention is described in detail below with reference to embodiments for purposes of exemplification and illustration only. Modifications to particular embodiments within the spirit and scope of the present invention, set forth in the appended claims, will be readily apparent to those of skill in the art.

### EXAMPLES

### Comparative Example A and Examples 1-3

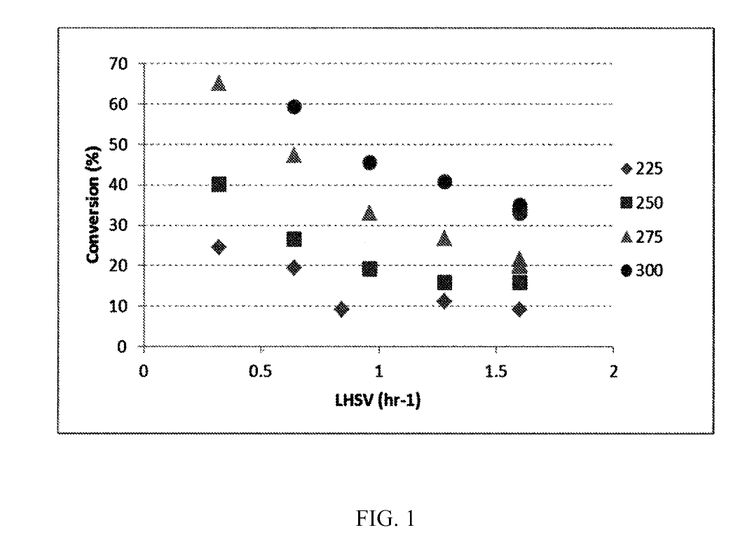

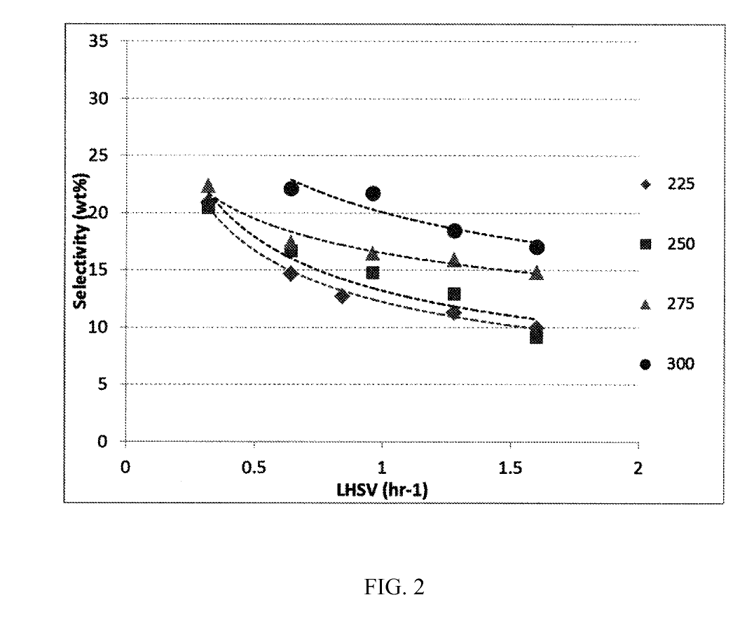

A feed stream comprising acetic acid was fed to a Berty reactor in the presence of a hydrogenation catalyst comprising platinum and tin on a modified silica support comprising calcium metasilicate. Comparative Example A was fed to the reactor at a temperature of 225° C. Example 1 was fed to the reactor at a temperature of 250° C. Example 2 was fed to the reactor at a temperature of 275° C. Example 3 was fed to the reactor at a temperature of 300° C. The acetic acid conversion of each example is shown in FIG. 1. Selectivity of acetic acid to ethyl acetate for Comparative Example A and Examples 1-3 is shown in FIG. 2.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

1. A process for producing ethanol, comprising contacting acetic acid and hydrogen in a reactor at a temperature of greater than 225° C. in the presence of a catalyst, under conditions effective to form ethanol, wherein the liquid hourly space velocity of the acetic acid fed to the reactor is from 0.3 to 1.6 hr⁻¹, wherein the catalyst comprises at least one metal selected from the group consisting of platinum, palladium, gold, iridium, osmium, and rhodium, on a support modified by a support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof.

2. The process of claim 1, wherein the temperature is from 225° C. to 300° C.

3. The process of claim 1, wherein the temperature is from 250° C. to 300° C.

4. The process of claim 1, wherein the liquid hourly space velocity of the acetic acid fed to the reactor is from 1 to 1.3 hr⁻¹.

5. The process of claim 1, wherein the temperature is at least 250° C. and the liquid hourly space velocity of the acetic acid fed to the reactor is from 0.5 to 1.6 hr⁻¹.

6. The process of claim 5, wherein conversion of acetic acid is at least 15%.

7. The process of claim 1, wherein the temperature is at least 275° C. and the liquid hourly space velocity of the acetic acid fed to the reactor is from 0.6 to 1.6 hr⁻¹.

8. The process of claim 7, wherein conversion of acetic acid is at least 20%.

9. The process of claim 1, wherein the temperature is at least 300° C. and the liquid hourly space velocity of the acetic acid fed to the reactor is from 1.2 to 1.6 hr⁻¹.

10. The process of claim 9, wherein conversion of acetic acid is at least 30%.

11. The process of claim 1, wherein acetic acid selectivity to ethanol is greater than 60%

12. The process of claim 1, wherein the catalyst further comprises at least one active metal selected from the group consisting of copper, iron, cobalt, vanadium, nickel, titanium, zinc, chromium, tin, lanthanum, cerium and manganese.

13. The process of claim 1, wherein the catalyst comprises platinum and tin.

14. The process of claim 1, wherein the support modifier is selected from the group consisting of oxides and metasilicates of sodium, potassium, magnesium, calcium, scandium, yttrium, and zinc.

15. The process of claim 1, wherein the support material is selected from the group consisting of silica, alumina, titania, silica/alumina, pyrogenic silica, high purity silica, zirconia, carbon, zeolites and mixtures thereof.

16. The process of claim 1, wherein the acetic acid is derived from a carbonaceous material selected from the group consisting of oil, coal, natural gas and biomass.

17. A process for producing ethanol, comprising contacting acetic acid and hydrogen in a reactor at a temperature from greater than 225° C. to 300° C. in the presence of a catalyst, under conditions effective to form ethanol wherein the liquid hourly space velocity of the acetic acid fed to the reactor is from 0.3 to 1.6 hr⁻¹, wherein the catalyst comprises a first metal selected from the group consisting of platinum, palladium, gold, iridium, osmium, and rhodium and a second metal selected from the group consisting of copper, iron, cobalt, vanadium, nickel, titanium, zinc, chromium, tin, lanthanum, cerium and manganese on a support.

18. The process of claim 17, wherein conversion of acetic acid is at least 15%.

19. The process of claim 17, wherein selectivity to ethanol is greater than 60%.

20. The process of claim 17, wherein the support is modified by a support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof.
